SEQUENCE LISTING (14 sequences)

SEQ ID NO: 1 - protein, 54 aa, Homo sapiens
MNSDSECPLSHDGYCLHDGVCMYIEALDKYACNCVVGYIGERCQYRDLKWWELR

SEQ ID NO: 2 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 3 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 4 - protein, 208 aa, Homo sapiens
MKLLPSVVLKLLLAAVLSALVTGESLEQLRRGLAAGTSNPDPSTGSTDQLLRLGGGRDRKVRDLQEADLDLLRVTLSSKPQALATPSKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 5 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 6 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 7 - protein, 27 aa, Artificial
  Artificial antagonist
SFYSCLESLVNGPAEKSRGQWDGCRKK

SEQ ID NO: 8 - protein, 54 aa, Homo sapiens
VCGDRGFYFNKPTGYGSSSRRAPQTGIVDECCFRSCDLRRLEMYCAPLKPAKSA

SEQ ID NO: 9 - protein, 83 aa, Artificial
  Artifiicial extended IGF-1
MFPAMPLSSLFVNGPETLCGAELVDALQFVCGDRGFYFNKPTGYGSSSRRAPQTGIVDECCFRSCDLRRLEMYCAPLKPAKSA

SEQ ID NO: 10 - protein, 83 aa, Artificial
  Artificial extended IGF-1
MFPAMPLSSLFVNGPGTLCGAELVDALQFVCGDRGFYFNKPTGYGSSSRRAPQTGIVDECCFRSCDLRRLEMYCAPLKPAKSA

SEQ ID NO: 11 - protein, 70 aa, Artificial
  Engineered mutant IGF-1
GPRTLCGAELVDALQFVCGDRGFYFNKPTGYGSSSRRAPQTGIVDECCFRSCDLRRLEMYCAPLKPAKSA

SEQ ID NO: 12 - protein, 67 aa, Homo sapiens
TLCGAELVDALQFVCGDRGFYFNKPTGYGSSSRRAPQTGIVDECCFRSCDLRRLEMYCAPLKPAKSA

SEQ ID NO: 13 - protein, 83 aa, Artificial
  Artificial extended IGF-1 variant
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 14 - protein, 71 aa, Artificial
  Artificial hybrid of insulin and IGF-1
FVNQHLCGSHLVEALYLVCGDRGFYFNKPTGYGSSSRRAPQTGIVDECCFRSCDLRRLEMYCAPLKPAKSA

(19) United States
(12) Patent Application Publication
(10) Pub. No.: US 2010/0197890 A1
(43) Pub. Date: Aug. 5, 2010

(54) Anti-cancer protein-platinum conjugates

(76) Inventor: Hugh McTavish, Birchwood, MN (US)

Correspondence Address: HUGH MCTAVISH;MCTAVISH PATENT FIRM, 429 BIRCHWOOD COURTS, BIRCHWOOD, MN, 55110, US

(21) Appl. No.: 12/657,906

(22) Filed: Jan. 29, 2010

Related U.S. Application Data
(60) Provisional application No. 61/206,360, filed on Jan. 31, 2009.

(51) Int. Cl. C07K 17/02; C07F 15/00

(52) U.S. Cl. 530/324; 556/137; 530/400

(57) ABSTRACT

The invention provides polypeptide-platinum conjugates comprising an anti-cancer platinum complex conjugated to polypeptides that bind relatively specifically to cancer cells, so as to direct the conjugates to cancer cells resulting in increased anti-cancer efficacy and decreased side-effects as compared to cisplatin and other conventional anti-cancer platinum complexes.

This application claims priority under 35 U.S.C. 119(e) from U.S. provisional patent application Ser. No. 61/206,360, filed Jan. 31, 2009.

### BACKGROUND

Cisplatin is among the most potent anti-cancer chemotherapy drugs available. It is widely used against cancers of the testis, ovary, bladder, head and neck, colon, and lung among other cancers. But the side effects of cisplatin are even more severe than many other chemotherapy drugs. It causes myelosuppression, nausea, neuropathy, and kidney toxicity, among other side effects. Newer platinum-based drugs carboplatin and oxaliplatin have been developed, but these also have systemic effects and side effect profiles that do not differ greatly from cisplatin.

The structure of cisplatin is shown below.

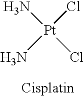

New chemotherapy agents with improved targeting to cancers are needed.

### SUMMARY

The invention provides novel platinum complexes that can be conjugated to proteins, conjugates of proteins and peptides with platinum complexes, methods of preparing the conjugates, and methods of treating cancer with the conjugates.

For instance, compound 11 is provided.

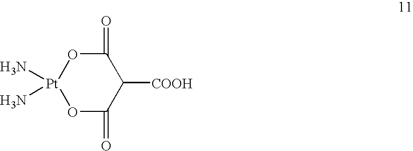

Complex 11 can be coupled to amino groups of proteins through the uncoordinated carboxyl group of the complex. The proteins are preferably proteins that can target the platinum complex more specifically to cancer cells, such as antibodies against receptor proteins found only or predominantly on cancer cells, or growth factors whose receptors are overexpressed on cancer cells.

Thus, one embodiment provides a platinum complex of formula I or II

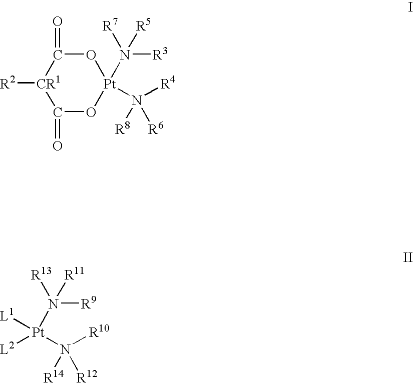

wherein in formula I

R¹ is H or (C₁-C₇)alkyl;

R² is COOH, NX₂, SH, HOOC—(C₁-C₁₀)alkyl, X₂N—(C₁-C₁₀)alkyl, HS—(C₁-C₁₀)alkyl, —CHO, OHC—(C₁-C₁₀)alkyl, or (C₁-C₆)alkyl-C(O)C(O)—(C₁-C₆)alkyl;

R³, R⁴, R⁵, R⁶, R⁷, and R⁸ are each independently H, (C₁-C₇)alkyl, or R³ and R⁴ together form (C₂-C₁₀)alkyl;
wherein in formula II

L¹ and L² are ligands selected from Cl⁻, formate, bicarbonate, NX₃, (C₁-C₁₀)alkyl-NX₂, and (C₁-C₁₀)alkyl-COO⁻, or L¹ and L² are together ⁻OOC—COO⁻, carboxy(C₁-C₁₀)alkyl-carboxy, X₂N—(C₁-C₁₀)alkyl-NX₂, or X₂N—(C₁-C₁₀)alkyl-carboxy;

R⁹, R¹⁰, R¹¹, R¹², R¹³, and R¹⁴ are each independently H, X₂N—(C₁-C₁₀)alkyl, HOOC—(C₁-C₁₀)alkyl, HS—(C₁-C₁₀)alkyl, —CHO, OHC—(C₁-C₁₀)alkyl, or (C₁-C₆)alkyl-C(O)C(O)—(C₁-C₆)alkyl; or R⁹ and R¹⁰ together are (C₂-C₁₀)alkyl, HOOC—(C₂-C₁₀)alkyl, X₂N—(C₂-C₁₀)alkyl, HS—(C₂-C₁₀)alkyl, OHC—(C₂-C₁₀)alkyl, or (C₁-C₆)alkyl-C(O)C(O)—(C₁-C₆)alkyl;

wherein at least one of R⁷, R⁸, R⁹, and R¹⁰ is HOOC—(C₁-C₁₀)alkyl, X₂N—(C₁-C₁₀)alkyl, HS—(C₁-C₁₀)alkyl, —CHO, OHC—(C₁-C₁₀)alkyl, or (C₁-C₆)alkyl-C(O)C(O)—(C₁-C₆)alkyl;

wherein L¹ is optionally R¹³ and L² is optionally R¹⁴;
wherein each X is independently H or (C₁-C₁₀)alkyl;
wherein each alkyl is optionally saturated or unsaturated, and straight chain, branched, or cyclic, optionally interrupted with —NH—, —O—, —S—, or ═N—, and optionally substituted with OH, halo, or oxo.

Another embodiment provides a polypeptide-platinum conjugate of formula III or IV

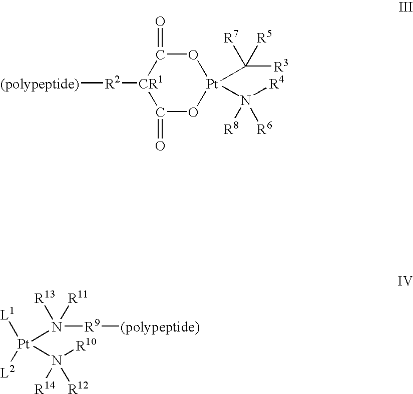

wherein in formula III

R¹ is H or (C₁-C₇)alkyl;

R² is a linker moiety of 1-100 atoms;

R³, R⁴, R⁵, R⁶, R⁷, and R⁸ are each independently H, (C₁-C₇)alkyl, or R³ and R⁴ together form (C₂-C₁₀)alkyl;
wherein in formula IV

L¹ and L² are ligands selected from Cl⁻, formate, bicarbonate, NX₃, (C₁-C₁₀)alkyl-NX₂, and (C₁-C₁₀)alkyl-COO⁻, or L¹ and L² are together ⁻COO—COO⁻, carboxy-(C₁-C₁₀)alkyl-carboxy, X₂N—(C₁-C₁₀)alkyl-NX₂, or X₂N—(C₁-C₁₀)alkyl-carboxy;

R⁹ is a linker moiety of 1-100 atoms;

R¹⁰, R¹¹, R¹², R¹³, and R¹⁴ are each independently H, (C₁-C₁₀)alkyl, X₂N—(C₁-C₁₀)alkyl, HOOC—(C₁-C₁₀)alkyl, or HS—(C₁-C₁₀)alkyl; or R¹¹ and R¹⁰ together are (C₂-C₁₀)alkyl, HOOC—(C₂-C₁₀)alkyl, X₂N—(C₂-C₁₀)alkyl, or HS—(C₂-C₁₀)alkyl; or R⁹ and R¹⁰ together are a linker moiety of 1-100 atoms;

wherein L¹ is optionally R¹³ and L² is optionally R¹⁴;
wherein each X is independently H or (C₁-C₁₀)alkyl;
wherein each alkyl is optionally saturated or unsaturated, and straight chain, branched, or cyclic, optionally interrupted with —NH—, —O—, —S—, or ═N—, and optionally substituted with OH, halo, or oxo.

Another embodiment provides a method of making a polypeptide-platinum conjugate comprising: forming a platinum complex as described above; and reacting the platinum complex with a linker reactant and a polypeptide to form a polypeptide-platinum conjugate.

Another embodiment provides a method of making a polypeptide-platinum conjugate comprising: reacting a platinum complex with a polypeptide-bidentate ligand conjugate of formula VI

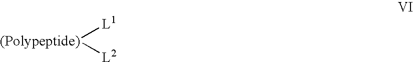

to form a polypeptide-platinum conjugate of formula VII

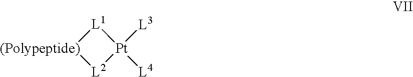

wherein L¹-L⁴ are each ligands. The polypeptide-platinum in particular embodiments is a conjugate of formula III or IV.

Another embodiment provides a method of making a polypeptide-platinum conjugate comprising: reacting a platinum complex with a polypeptide-ligand conjugate of formula VIb

(Polypeptide)-L¹ VIb

to form a polypeptide-platinum conjugate of formula VIIb

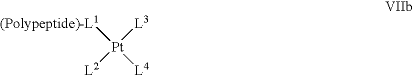

wherein L¹-L⁴ are each ligands. Preferably the polypeptide-platinum conjugate of formula VIIb is a polypeptide-platinum conjugate formula III or IV.

Another embodiment provides a polypeptide-platinum complex of formula X

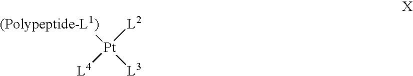

wherein L¹ is an amino, caboxy, or sulfhydryl group of the polypeptide that is a ligand to the Pt, and L²-L⁴ are ligands.

Another embodiment provides a method of treating cancer comprising administering a polypeptide-platinum conjugate of formula III, IV, VII, VIIb, or X to a mammal afflicted with cancer.

### DETAILED DESCRIPTION

### Definitions

The term “binding affinity” of a ligand for a particular receptor refers to the association constant K_{A} (the inverse of the dissociation constant K_{D}) or to experimentally determined approximations thereof.

The term “agonist” refers to a ligand to a receptor (for instance, the insulin receptor, type 1 IGF receptor, or EGF receptor) that, when it binds to the receptor, activates the normal biochemical and physiological events triggered by binding of the natural ligand for the receptor (i.e, insulin for the insulin receptor, IGF-1 for the IGF-1 receptor, or EGF for the EGF receptor). In particular embodiments, an agonist has at least 20%, at least 30%, or at least 50% of the biological activity of the natural ligand. The activity of an insulin receptor ligand can be measured, for instance, by measuring the hypoglycemic effect (Poznansky, M. J., et al., 1984, Science 223:1304). The activity of an insulin-receptor ligand or IGF-1-receptor ligand can be measured in vitro by the measuring the extent of autophosphorylation of the receptor in response to ligand binding, as described in Satyamarthy, K., et al., 2001, Cancer Res. 61:7318. MAP kinase phosphorylation can also be measured for the IGF-1 receptor (Satyamarthy, K., et al., 2001, Cancer Res. 61:7318). EGF receptor tyrosine kinase activity can be assayed as described in Beerli, R. R., et al., 1996, J. Biol. Chem. 271:6071-6076.

The term “antagonist” refers to a ligand that has little or no stimulating activity when it binds to the receptor and that competes with or inhibits binding of the natural ligand to the receptor. In particular embodiments, an antagonist has less than 20%, less than 10%, or less than 5% of the activity of the natural ligand (insulin for the insulin receptor or IGF-1 for the IGF-1 receptor).

Alkyls are described herein as being optionally saturated or unsaturated, straight chain, branched, or cyclic, and optionally interrupted with —NH—, —O—, —S—, or ═N—, and optionally substituted with OH, halo, or oxo. Thus, for instance, a “(C₁-C₁₀)alkyl,” may be a heteroaryl ring.

### DESCRIPTION

The embodiments of the invention are directed to polypeptide-platinum conjugates suitable for treating cancer, and methods of making them. Cisplatin is one of the most effective anti-cancer chemotherapy drugs, but has the drawback of causing extreme side effects. Several other platinum complexes with anti-cancer properties have been investigated (references 1-4), including carboplatin and oxaliplatin, but like cisplatin, they are not directed specifically to cancer cells, but instead are taken up by all cells in the body. Therefore, they have similarly extreme systemic side effects.

The aim of the invention is to develop conjugates containing anti-cancer platinum complexes attached to proteins or peptides that bind at least somewhat specifically to cancer cells. Examples of suitable proteins include growth factors or hormones whose receptors are overexpressed on cancer cells. The epidermal growth factor (EGF) receptor, the type I insulin-like growth factor receptor, and the insulin receptor are all overexpressed on many if not most types of cancer. Thus, ligands to these receptors, or other polypeptides that bind somewhat specifically to cancer cells, may be attached to platinum complexes to deliver the platinum complexes more specifically to cancer cells. The polypeptide ligands are preferably internalized by the cells when they bind to their receptors. That way, the platinum complex is also internalized efficiently to the cancer cells. Agonists are internalized, while antagonists in some cases are not.

The conjugates are formed by creating a platinum complex that has at least one ligand with a free group that is chemically suitable for cross-linking to a polypeptide. Such groups include carboxyl, amino, and mercapto groups, as well as aldehyde groups and di-ketone groups. Cross-linkers exist that can react with carboxyl and amino groups, for instance, to cross-link them to each other. Thus, a platinum complex with a free carboxyl group can be cross-linked to an amino group on a protein, e.g., a lysine side chain, to form a polypeptide-platinum complex. Alternatively, a free ligand molecule can be cross-linked to a protein, and then used to ligate platinum and form a polypeptide-platinum complex.

For instance, CH(COOH)₃ can be used to ligate platinum to form platinum complex 11 where two of the three carboxyls of CH(COOH)₃ ligate platinum, and one carboxyl is free to react with a cross-linker.

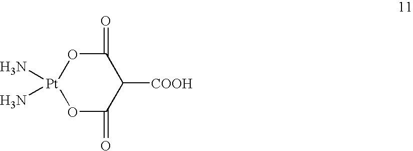

Alternatively, CH(COOH)₃ can be first cross-linked to a protein, and then the resultant (protein-NH)—CO—CH(COOH)₂ conjugate can ligate a platinum atom in a complex to form the same protein-platinum conjugate.

For another example the bidentate ligand H₂NCH(COOH)₂ can be coupled to a polypeptide by a bifunctional cross-linking reagent that reacts with amino groups to cross-link the ligand through its amino group to a an amino group of polypeptide to form the conjugate (polypeptide-NH)-linker-NH—CH(COOH)₂ and the conjugate can then ligate a platinum atom through the two carboxyls of the conjugate.

### Guidelines for Coupling Ligands or Platinum Complexes to Polypeptides

The platinum complexes of the invention are typically coupled to polypeptides through the reactive groups present on proteins. These include the N-terminal alpha-amino group, the C-terminal alpha-carboxyl group, the side-chain amino group of lysine, the side-chain carboxyl groups of aspartic acid and glutamic acid, the side chain thiol of cysteine, and the side chain of arginine. Other reactive side chains found on proteins are the side-chain hydroxyl of serine and threonine, the hydroxyaryl of tyrosine, the imidazole of histidine, and the methionine side chain. But the predominant reactive groups are amino, carboxyl, and mercapto groups found on amino acid side chains and the amino and carboxyl terminus of a polypeptide.

In the embodiments of the invention, the same reactive groups are placed on ligands to platinum, preferably bidentate ligands to platinum, and the ligand reactive groups are cross-linked to the reactive groups of the polypeptides. Thus, cross-linking a ligand or platinum complex to a polypeptide is analogous to cross-linking two polypeptides.

The chemistry and principles of protein conjugation and cross-linking are described in Wong, Shan S., Chemistry of Protein Conjugation and Cross-Linking, 1991, CRC Press, Boca Raton, Fla. Other sources for information on this chemistry include the Pierce Biochemistry catalog; and Greene, T. W., and Wutz, P. G. M., Protecting Groups in Organic Synthesis, second edition 1991, John Wiley & Sons, Inc., New York, and references cited therein.

The strongest nucleophile of amino acid side chains is the thiol of reduced cysteine side chains. The thiol reacts with most protein modifying reagents. Alpha-haloacetamides and maleimides are considered to react specifically with cysteine residues, particularly at pH 7.0 and below. Thiols also react by disulfide interchange with disulfide reagents.

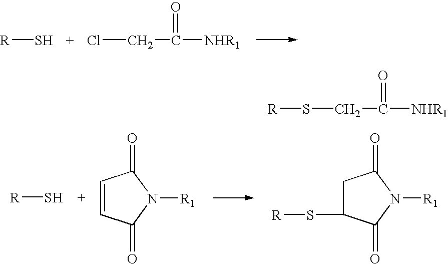

Amino groups are the next-strongest nucleophiles found on proteins. Aldehydes react with amino groups to form Schiff bases. The Schiff bases are hydrolyzable, which can be an advantage in the present invention. With uptake into cancer cells of a ligand-chemotherapeutic agent conjugate, in some cases it is necessary that the chemotherapeutic agent is cleaved from the conjugate for it to be active. This is better accomplished if the chemotherapeutic agent is linked to the ligand by a cleavable linkage, such as a hydrolyzable linkage. Cleavable linkages can be cleaved spontaneously or by enzymes in the cell. For instance, amide bonds are cleaved by certain enzymes, including proteases. A Schiff base linkage spontaneously hydrolyzes at an appreciable rate. A disulfide linkage is expected to be reductively cleaved in the intracellular reducing environment of a cancer cell.

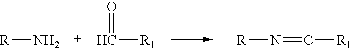

The Schiff base formed by reaction of an amino group with an aldehyde can be stabilized by reduction with, for instance, sodium borohydride or pyridine borane. Pyridine borane has the advantage of not reducing disulfides, which are found in insulin, IGF-1, and IGF-2 and are essential for the structure of those proteins.

A dialdehyde, such as glutaraldehyde, will cross-link two molecules having amino groups.

Other amino reagents include activated carbonyls, such as N-hydroxysuccinimide esters, p-nitrophenyl esters, or acid anhydrides (e.g., succinic anhydride).

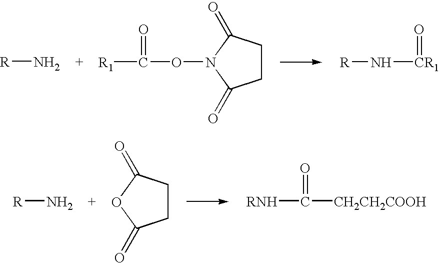

Amino groups also react with sulfonyl halides and aryl halides (e.g, 2,4-dinitrofluorobenzene).

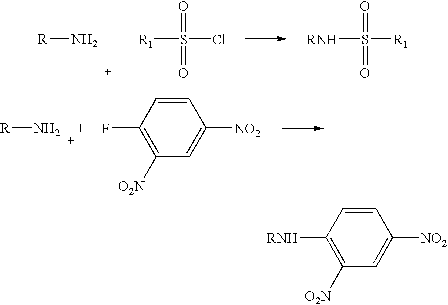

Amino groups also react with isocyanates and isothiocyanates to form urea or thiourea derivatives.

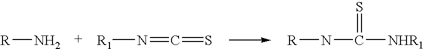

Imidoesters are the most specific acylating agents for amino groups. Imidoesters react specifically with amines to from imidoamides at pHs between about 7 and 10. This reaction has the advantage of maintaining charge stability by generating a positively charged group, the imidoamide, at the former amino group. Imidoamides also slowly hydrolyze at pHs above neutrality, which can also be an advantage in that the hydrolysis can release free chemotherapeutic agent in the cancer cell.

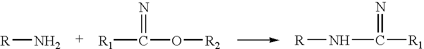

Carboxyl groups react specifically with diazoacetate and diazoacetamide under mild acid conditions, e.g., pH 5.

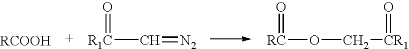

The most important chemical modification of carboxyls uses carbodiimides, such as 1-cyclohexyl-3-(2-morpholinyl-4-ethyl)carbodiimide (CMC) and 3-(3-dimethylaminopropyl)carbodiimide (EDC). In the presence of an amine, carbodiimides form an amide bond to the carboxyl in two steps. In the first step, the carboxyl group adds to the carbodiimide to form an O-acylisourea intermediate. Subsequent reaction with an amine yields the corresponding amide.

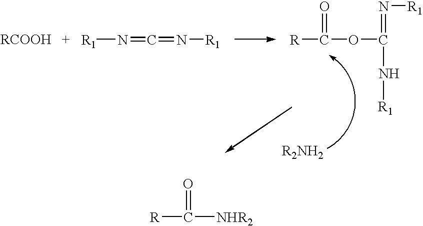

A particularly important carbodiimide reaction is its use in activating carboxyls with N-hydroxysuccinimide to form an N-hydroxysuccinimide ester.

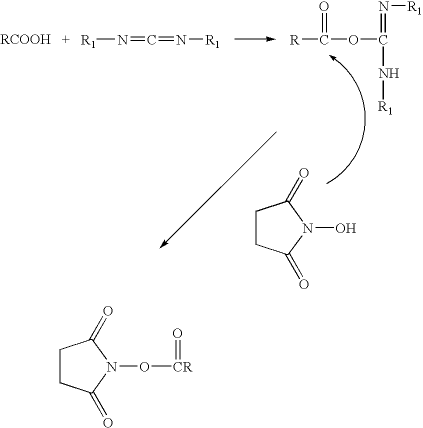

The activated carboxyl is stable enough to be isolated, but will then readily react with amino groups to form an amide bond.

Succinimides such as N-succinimidyl-3-[2-pyridyldithio]propionate (SPDP) can be used to couple two compounds through amino groups. (See Pierce Biotechnology catalog, and Thorpe, P. E. et al. 1982, Immunol. Rev. 62:119-158.)

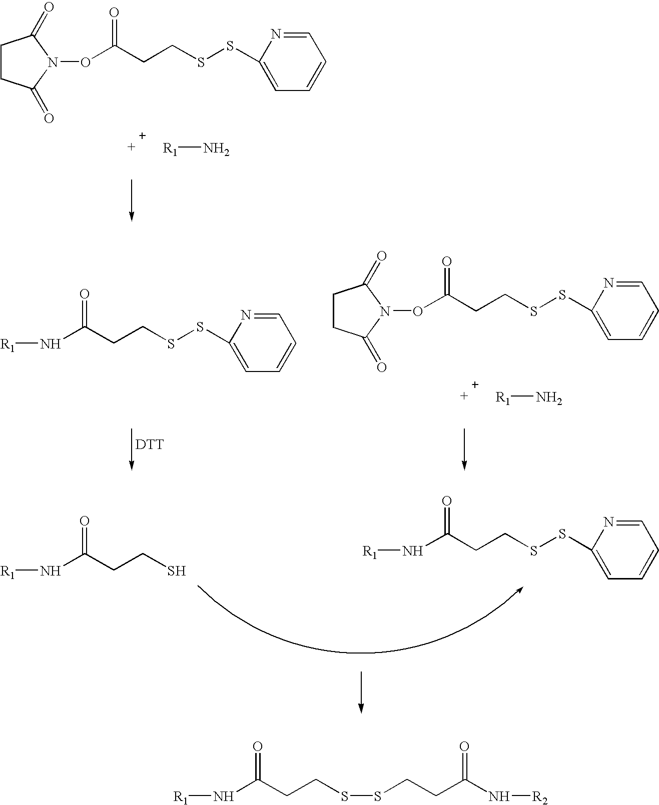

Arginine reacts with vicinal dialdehydes or diketones, such as glyoxal, 2,3-butanedione, and 1,2-cyclohexanedione. Borate may stabilize the adduct, if stabilization is desired.

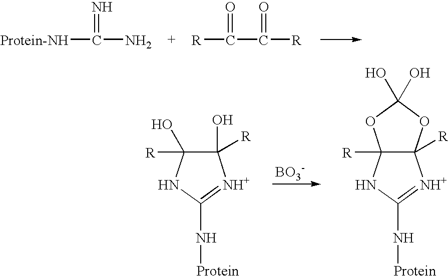

The reactive groups can also be interchanged with other reactive groups by some of the above reactions. For instance, modification of an amino group with an acid anhydride such as succinic anhydride, replaces the positively charged amino group with a free carboxyl group. Likewise, reaction of a carboxyl group with a carbodiimide and a diamine, such as ethylene diamine, replaces the carboxyl group with a free amino group.

Cross-linking: Reagents containing two of the reactive groups described above, for instance two amino-reactive groups or an amino-reactive and a thiol-reactive group, can be used to cross-link a platinum complex (or ligand that can be complexed to platinum) containing one of the appropriate groups, particularly carboxyl, amino, or mercapto, to a polypeptide containing the other appropriate group. For instance, a platinum complex containing a free amino group can be cross-linked to an amino group (lysine side chain or N-terminal amino) of a polypeptide by a cross-linker having two amine-reactive groups. For example, an free amino on a platinum complex or platinum ligand can be coupled to an amino on a polypeptide by a di-imidoester, such as dimethyladipimidate-2-HCl (Pierce Biochemical, Inc.), or a disuccinimidyl ester, such as disuccinimidyl glutarate (Pierce Biochemical, Inc.).

A carboxyl (e.g., a free carboxyl of a platinum complex) can be activated with a carbodiimide or a carbodiimide and N-hydroxysuccinimide to react with an amino group (of, e.g., a protein ligand) to form an amide bond cross-link.

Where ligands or reagents are not commercially available, they can be synthesized by principles and procedures known to organic chemists and described in references 5-9.

### Specific Embodiments

In particular embodiments of the platinum complexes of formula I or polypeptide-platinum conjugates of formula III R³-R⁸ are each H. In other embodiments, R³ and R⁴ together form (C₂-C₃)alkyl, and R⁵-R⁸ are each H.

In some embodiments of the platinum complexes of formula I or polypeptide-platinum conjugates of formula III, R³ and R⁴ together form (C₂-C₃)alkyl, optionally substituted with (C₁-C₁₀)alkyl, wherein both alkyls are optionally interrupted with —NH—, —O—, —S—, or ═N—, and optionally substituted with OH, halo, or oxo, amino, carboxy, or mercapto. In this case, the two amines ligating the platinum are joined together and form a bidentate ligand.

Likewise, in particular embodiments of the complex of formula II R⁹ and R¹⁰ together form (C₂-C₃)alkyl, optionally substituted with (C₁-C₁₀)alkyl, wherein both alkyls are optionally interrupted with —NH—, —O—, —S—, or ═N—, and optionally substituted with OH, halo, or oxo, amino, carboxy, or mercapto. In this case, the two amines ligating the platinum are joined together and form a bidentate ligand.

In some embodiments, R¹¹ and R¹² are each H, and R⁹ and R¹⁰ together form —(C₂-C₃)alkyl- optionally substituted with carboxy, amino, mercapto, carboxy(C₁-C₄)alkyl, amino(C₁-C₄)alkyl, or mercapto(C₁-C₄)alkyl; and R₁₃ and R₁₄ are independently H, carboxy(C₁-C₄)alkyl, amino(C₁-C₄)alkyl, or mercapto(C₁-C₄)alkyl.

In other embodiments of the complex of formula II, R⁹ and R¹⁰ together form —(C₂-C₃)alkyl- optionally substituted with carboxy or carboxy(C₁-C₄)alkyl; and R¹¹-R¹⁴ are each independently H, (C₁-C₄)alkyl, or carboxy(C₁-C₄)alkyl; wherein at least one of R⁹-R¹⁴ is carboxy(C₁-C₆)alkyl.

In particular embodiments of the polypeptide-platinum conjugates, the complex is linked to the polypeptide by an amide bond. Thus, the linker moiety comprises a —C(═O)— or —NH— portion of amide bond. In other embodiments, the complex is linked to the polypeptide by a disulfide bond or a Schiff base or a reduced Schiff base.

In particular embodiments of the method of making a polypeptide-platinum conjugate the method comprises: reacting a platinum complex of formula V with a polypeptide-bidentate ligand conjugate of formula VI

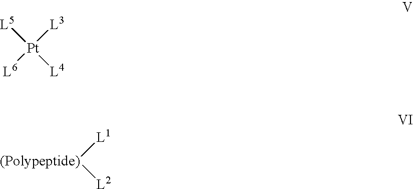

to form a polypeptide-platinum conjugate of formula VII

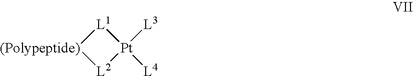

wherein L¹-L⁶ are each ligands.

In particular embodiments, the polypeptide-bidentate ligand conjugate of formula VI is a conjugate of formula VIII

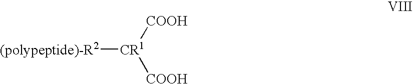

wherein R¹ is H or (C₁-C₇)alkyl and R² is a linker moiety of 1-100 atoms;

wherein each alkyl is optionally saturated or unsaturated, and straight chain, branched, or cyclic, optionally interrupted with —NH—, —O—, —S—, or ═N—, and optionally substituted with OH, halo, or oxo.

In other embodiments, the polypeptide-bidentate ligand conjugate of formula VI is a conjugate of formula IX

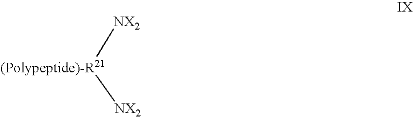

wherein R²¹ is (C₁-C₂)alkyl, optionally substituted with (C₁-C₇)alkyl, and each X is independently H or (C₁-C₁₀)alkyl; wherein each alkyl is optionally saturated or unsaturated, and straight chain, branched, or cyclic, optionally interrupted with —NH—, —O—, —S—, or ═N—, and optionally substituted with OH, halo, or oxo.

The polypeptide-platinum conjugate of formula VII may undergo further ligand substitution to arrive at a final product for treating cancer. For instance, where L¹ and L² are a bidentate diamine ligand, L³ and L⁴ may be iodides, and the iodides may be substituted in a later step with, for instance, chlorides, oxalate, or malonate.

### Polypeptides

Examples of proteins suitable to conjugate to platinum complexes include growth factors or hormones whose receptors are overexpressed on cancer cells. The epidermal growth factor (EGF) receptor, the type I insulin-like growth factor receptor, and the insulin receptor are all overexpressed on many if not most types of cancer. Thus, ligands to these receptors, or other polypeptides that bind somewhat specifically to cancer cells, may be attached to platinum complexes to deliver the platinum complexes more specifically to cancer cells. The polypeptide ligands are preferably internalized by the cells when they bind to their receptors. That way, the platinum complex is also internalized efficiently to the cancer cells. Agonists are internalized, while antagonists in some cases are not.

Insulin of course is the natural ligand for the insulin receptor. Insulin-like growth factor 1 (IGF-1) is the natural ligand for the type I IGF receptor. Insulin and IGF-1 also cross-react with each other's receptors, and IGF-2 binds to both receptors as well.

Another receptor often found in greater numbers in cancer cells than in normal cells of the same tissue type is the epidermal growth factor (EGF) receptor. (Nicholson, R. I. et al., 2001, Eur. J. Cancer 37:S9-S15. Kopp, R., et al., 2003, Recent Results in Cancer Research 162:115-132. Fox, S. B. et al., 1994, Breast Cancer Res. Treat. 29:41-49. The EGF receptor, also known as ErbB-1, is activated by several agonists, including EGF itself, transforming growth factor alpha (TGFα), amphiregulin (AR), heparin-binding EGF-like growth factor (HB-EGF), and betacellulin (BTC). (Beerli, R. R. et al., 1996, J. Biol. Chem. 271:6071-6076. Earp, H. S., et al., 2003, Trans. Am. Clin. Clim. Assoc. 114:315-333.) Three other receptors are also considered members of the EGF family of receptors. They are ErbB-2, ErbB-3, and ErbB-4 (also known as HER2, HER3, and HER4, for human EGF receptor 2, 3, and 4, respectively). These receptors, especially ErbB-2, are also often overexpressed on cancerous cells. The receptors ErbB-2 and ErbB-4 are tyrosine kinases. The EGF receptor agonists listed above bind most strongly to the EGF receptor. They bind less tightly to the other receptors in the EGF receptor family. Neu differentiation factors (NDFs)/heregulins are ligands for EbrB-3 and ErbB-4. (Beerli, R. R., 1996J. Biol. Chem. 271:6071-6076. Carraway, K. L. et al., 1994, J. Biol. Chem. 269:14303-14306. Plowman, G. D., et al., 1993, Nature 366:473-475.)

Thus, EGF, TGFα, HB-EGF, BTC, and NDFs are also proteins that may be coupled to platinum complexes.

Peptide libraries may also be screened, e.g., by phage display library techniques, to identify nonnatural peptides that bind to one of the target receptor proteins overexpressed on cancer cells, including the insulin receptor, IGF-1 receptor, EGF receptor, and ErbB-2. These peptides can be conjugated to platinum complexes as described herein.

Antibodies against these receptors or other targets that are relatively specific for cancer cells can also be conjugated to the platinum complexes. Antibodies against CA125 are an example.

Thus, the polypeptides can be any size, from short chemically synthesized peptides to large multi-subunit proteins.

Another particular polypeptide for conjugation to a platinum complex is a variant of IGF-1 that has reduced binding to the type I IGF receptor.

In particular embodiments, the polypeptide is a ligand to the insulin receptor, IGF-1 receptor, EGF receptor, or Erb-2.

The sequence of a precursor of EGF is SEQ ID NO:1. In mature EGF, the amino terminal methionine of SEQ ID NO:1 is removed. (Gregory, H., 1975, Nature 257:325-327.) The sequence of the precursor of TGFα is SEQ ID NO:2. Mature TGFα is thought to be residues 40-89 of SEQ ID NO:2. (Qian, J. F., et al., 1993, Gene 132:291-296. Higashayaam, S., et al., 1991, Science 251:936-939.) The sequence of the precursor of amphiregulin is SEQ ID NO:3. Mature amphiregulin is thought to be residues 101-184 of SEQ ID NO:3. (Plowman, G. D., et al., 1990, Mol. Cell. Biol. 10:1969-1981.) The sequence of the precursor of HB-EGF is SEQ ID NO:4. Mature HB-EGF is thought to be residues 63-148 of SEQ ID NO:4. (Higashayama, S. et al., 1992, J. Biol. Chem. 267:6205-6212. Higashayaam, S., et al., 1991, Science 251:936-939.) The sequence of the precursor of betacellulin is SEQ ID NO:5. Mature betacellulin is thought to be residues 32-111 of SEQ ID NO:5. (Sasada, R. et al., 1993, Biochem. Biophys. Res. Comm. 190:1173-1179.) Cysteine residues 7 with 21, 15 with 32, and 34 with 43 of SEQ ID NO:1 form disulfide bridges to each other in mature EGF. (Gregory, H., 1975, Nature 257:325-327.) The homologous cysteine residues in the other natural EGF receptor ligands also form disulfide bridges. (Higashayaam, S., et al., 1991, Science 251:936-939.) Another polypeptide ligand to the EGF receptor is a chimera of sequences from natural EGF receptor ligands, e.g., the chimera E4T, which is a chimera of EGF and TGFαsequences, and is a more active agonist than either EGF or TGFα. (Lenferink, A. E. G., et al., 1998, EMBO J. 17:3385-3397. Kramer, R. H., et al., 1994, J. Biol. Chem. 269:8708-8711.)

In particular embodiments, the polypeptide is a ligand to the EGF receptor, the ligand comprising a polypeptide sequence selected from the group consisting of residues 2-54 of SEQ ID NO:1, residues 40-89 of SEQ ID NO:2, residues 101-184 of SEQ ID NO:3, residues 63-148 of SEQ ID NO:4, residues 32-111 of SEQ ID NO:5, and E4T.

The structure of insulin is well known and is disclosed in U.S. published patent application 20060258569. The amino acid sequence of IGF-1 is SEQ ID NO:6.

Examples of agonist and antagonist peptide ligands to the IGF-1 receptor, and methods of identifying agonist and antagonist peptide ligands to the IGF-1 receptor, are disclosed in U.S. published patent applications 2004/0023887 and 2003/0092631. One antagonist is the peptide SFYSCLESLVNGPAEKSRGQWDGCRKK (SEQ ID NO:7).

Other examples of IGF-1 receptor agonists include variants of IGF-1 that activate the receptor but have reduced affinity for the soluble IGF-1 binding proteins disclosed in U.S. Pat. No. 4,876,242. IGF binding proteins are natural serum proteins that bind to IGF-1, holding it in circulation and extending its biological half-life. It may be advantageous for the IGF-1 receptor ligands of this invention, particularly agonists co-administered with chemotherapeutic agents as separate molecules, to have reduced binding to the IGF-1 binding proteins, because that reduced binding would accelerate the release of the agent to bind to the IGF-1 receptors. Thus, in some embodiments, the IGF-1 receptor ligand or agonist has reduced affinity for soluble IGF-1 binding proteins, as compared to native IGF-1. Variants disclosed in U.S. Pat. No. 4,876,242 include variants wherein the variant IGF-1 comprises the polypeptide structure A₁-A₂-A₃-A₄-LCG-A₅-A₆-LV-A₇-AL-A₈-A₉-R₁, wherein A₁ is G, V, or FV; A₂ is P or N; A₃ is E or Q; A₄ is T, H, or A; A₅ is A or S; A₆ is E or H; A₇ is D or E; A₈ is Q or Y; A₉ is F or L; and R₁ is SEQ ID NO:8. In a specific embodiment, A₁ is FV, A₂ is N, A₃ is Q, A₄ is H, A₅ is S, A₆ is H, A₇ is E, A₈ is Y, and A₉ is L, and thus the variant is SEQ ID NO:14. In other embodiments, the variant comprises SEQ ID NO:14 or another variant disclosed in U.S. Pat. No. 4,876,242. In other embodiments, the variant comprises SEQ ID NO:8.

One preferred variant IGF-1 with reduced binding to the soluble IGF binding proteins, for use in the methods and conjugates of the invention is LONG-R3-IGF-1 (SEQ ID NO:13) (Francis, G. L., et al. 1992, J. Mol. Endocrinol. 8:213-223; Tomas, F. M. et al., 1993, J. Endocrinol. 137:413-421). Other variant IGF-1s that have reduced affinity for the soluble IGF-1 binding proteins include SEQ ID NOS:9-12, especially Des(1-3)IGF-1, SEQ ID NO:12, which lacks the first 3 residues of wild-type IGF-1. Thus, in particular embodiments, the polypeptide that is a variant IGF-1 with reduced binding to the soluble IGF-1 binding proteins comprises any one of SEQ ID NOS:9-13.

Preferably, the IGF-1 receptor ligand with reduced affinity for soluble IGF-1 binding proteins has at least 5-fold, more preferably at least 10-fold, more preferably still at least 100-fold lower binding affinity for soluble IGF-1 binding proteins than wild-type IGF-1. Binding affinity for the soluble IGF-1 binding proteins can be measured by a competition binding assay against labeled IGF-1 (e.g., I-125-IGF-1), using a mixture of purified IGF-1 binding proteins or rat L6 myoblast-conditioned medium (a naturally produced mixture of IGF-1 binding proteins), as described in Francis, G. L., et al. (1992, J. Mol. Endocrinol. 8:213-223) and Szabo, L. et al. (1988, Biochem. Biophys. Res. Commun. 151:207-214). Preferably, the variant IGF-1 has an IC₅₀ in a competition binding assay against labeled wild-type IGF-1 for binding to soluble IGF-1 binding proteins in L6 myoblast-conditioned medium of greater than 10 nM, more preferably greater than 100 nM.

Preferably, the variant IGF-1 with reduced affinity for soluble IGF-1 binding proteins has affinity for the IGF-1 receptor that is close to wild-type IGF-1 (e.g., less than 30-fold greater than wild-type IGF-1, more preferably less than 10-fold greater than wild-type IGF-1). In specific embodiments, the variant IGF-1 has an IC₅₀ in a competition binding assay against labeled wild-type IGF-1 for binding to IGF-1 receptors (e.g., on MCF-7 cells) of less than 50 nM, more preferably less than 10 nM, more preferably still less than 5 nM, more preferably still less than 3 nM). This assay is described in Ross, M. et al. (1989, Biochem. J. 258:267-272) and Francis, G. L., et al. (1992, J. Mol. Endocrinol. 8:213-223).

Preferably, the polypeptide and/or the polypeptide-platinum conjugate has a K_{D} for its target receptor or target molecule that is somewhat specific for cancer cells of less than 10 μM, less than 1 μM, less than 100 nM, less than 50 nM, less than 20 nM, less than 10 nM, less than 5 nM, less than 2 nM, or less than 1 nM.

Most cytokines are not extremely soluble in neutral aqueous solution. Thus, to make them more soluble, they can be expressed as fusion proteins with a more soluble sequence such as all or part of serum albumin. Thus, in some embodiments, the polypeptide is a fusion protein comprising a all or a portion of a cytokine and all or a portion of another polypeptide sequence.

EGF precursor:

(SEQ ID NO: 1)

MNSDSECPLS HDGYCLHDGV CMYIEALDKY ACNCVVGYIG
ERCQYRDLKW WELR
TGFα precursor

(SEQ ID NO: 2)

MVPSAGQLAL FALGIVLAAC QALENSTSPL SADPPVAAAV
VSHFNDCPDS HTQFCFHGTC RELVQEDKPA CVCHSGYVGA
RCEHADLLAV VAASQKKQAI TALVVVSIVA LAVLIITCVL
IHCCQVRKHC EWCRALICRH EKPSALLKGR TACCHSETVV
Amphiregulin precursor:

(SEQ ID NO: 3)

MRAPLLPPAP VVLSLLILGS GHYAAGLDLN DTYSGKREPF
SGDHSADGFE VTSRSEMSSG SEISPVSEMP SSSEPSSGAD
YDYSEEYDNE PQIPGYIVDD SVRVEQVVKP PQNKTESENT
SDKPKRKKKG GKNGKNRRNR KKKNPCNAEF QNFCIHGECK
YIEHLEAVTC KCQQEYFGER CGEKSMKTHS MIDSSLSKIA
LAAIAAFMSA VILTAVAVIT VQLRRQYVRK YEGEAEERKK
LRQENGNVHA IA
HD-EGF precursor

(SEQ ID NO: 4)

MKLLPSVVLK LLLAAVLSAL VTGESLEQLR RGLAAGTSNP
DPSTGSTDQL LRLGGGRDRK VRDLQEADLD LLRVTLSSKP
QALATPSKEE HGKRKKKGKG LGKKRDPCLR KYKDFCIHGE
CKYVKELRAP SCICHPGYHG ERCHGLSLPV ENRLYTYDHT
TILAVVAVVL SSVCLLVIVG LLMFRYHRRG GYDVENEEKV
KLGMTNSH
Betacellulin precursor:

(SEQ ID NO: 5)

MDRAARCSGA SSLPLLLALA LGLVILHCVV ADGNSTRSPE
TNGLLCGDPE ENCAATTTQS KRKGHFSRCP KQYKHYCIKG
RCRFVVAEQT PSCVCDEGYI GARCERVDLF YLRGDRGQIL
VICLIAVMVV FIILVIGVCT CCHPLRKRRK RKKKEEEMET
LGKDITPINE DIEETNIA
IGF-1:

(SEQ ID NO: 6)

GPETLCGAELVDALQFVCGDRGFYFNKPTGYGSSSRRAPQT
GIVDECCFRSCDLRRLEMYCAPLKPAKSA

SEQ ID NO: 7
(SEQ ID NO: 7)

SFYSCLESLVNGPAEKSRGQWDGCRKK.
SEQ ID NO: 8:

(SEQ ID NO: 8)

VCGDRGFYFN KPTGYGSSSR RAPQTGIVDE CCFRSCDLRR
LEMYCAPLKP AKSA
Long-IGF-1

(SEQ ID NO: 9)

MFPAMPLSSL FVNGPETLCG AELVDALQFV CGDRGFYFNK
PTGYGSSSRR APQTGIVDEC CFRSCDLRRL EMYCAPLKPA KSA
Long-Gly3-IGF1

(SEQ ID NO: 10)

MFPAMPLSSL FVNGPGTLCG AELVDALQFV CGDRGFYFNK
PTGYGSSSRR APQTGIVDEC CFRSCDLRRL EMYCAPLKPA KSA
R3-IGF1

(SEQ ID NO: 11)

GPRTLCGAEL VDALQFVCGD RGFYFNKPTG YGSSSRRAPQ
TGIVDECCFR SCDLRRLEMY CAPLKPAKSA
Des (1-3)-IGF1

(SEQ ID NO: 12)

TLCGAELVDA LQFVCGDRGF YFNKPTGYGS SSRRAPQTGI
VDECCFRSCD LRRLEMYCAP LKPAKSA
Long-R3-IGF1:

(SEQ ID NO: 13)

MFPAMPLSSL FVNGPRTLCG AELVDALQFV CGDRGFYFNK
PTGYGSSSRR APQTGIVDEC CFRSCDLRRL EMYCAPLKPA KSA
Insulin-IGF1 hybrid

(SEQ ID NO: 14)

FVNQHLCGSHLVEALYL VCGDRGFYFN KPTGYGSSSR
RAPQTGIVDE CCFRSCDLRR LEMYCAPLKP AKSA

### EXAMPLES

### Example 1

All procedures are carried out in darkness or dim light to avoid formation of iodoplatinum precipitates.

K₂PtI₄ formation. A solution of 5 g (12 mmol) K₂PtCl₄ is treated with KI (12 g, 72 mmol) in 18 ml water, heated to 70° C., and allowed to cooled (0.5 hours). The product K₂PtI₄ is filtered.

Cis(NH₃)₂PtI₂ formation. The filtered K₂PtI₄ in solution is treated with 12-13 ml 2.0 M NH₃. After 30 minutes, the product cis(NH₃)₂PtI₂ is filtered, washed with cold water, and dried in a dessicator.

Cis(NH₃)₂Pt(CH(COO)₃) formation. Cis(NH₃)₂PtI₂ is stirred with 2 mole equivalents of silver nitrate overnight in aqueous solution. AgI is then filtered out. The filtrate contains product cis-[(NH₃)₂Pt(OH₂)₂](NO₃)₂.

cis-[(NH₃)₂Pt(OH₂)₂](NO₃)₂. is mixed with 1 mole equivalent of K₃CH(COO)₃. The solution is allowed to stand for 24 hours and then evaporated to dryness under vacuum. The product is complex 11:

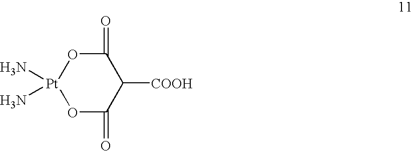

The product mixture also include potassium nitrate.

Protein conjugation: Platinum complex 11 (30 μmoles) is dissolved with insulin (2 μmoles) in 3.4 ml of 20 mM sodium phosphate, pH 7.4, 6.5 M urea. The cross-linker 1-ethyl-3-[3-dimethylaminopropyl]carbodiimide hydrochloride (EDC) (300 μmoles) is freshly dissolved in 0.6 ml of the buffer, and added to the protein-complex 11 solution. The solution is allowed to react for 2 hours at room temperature and then placed in a dialysis bag (3,500 m.w. cut-off). The solution is dialyzed three hours against 20 mM sodium phosphate, pH 7.4, 6.5 M urea, and then dialyzed overnight against 2 mM NaOH.

The product is an insulin-platinum conjugate with approximately 3 complex 11 per insulin conjugated by direct amide bonds between amino groups of insulin and the free carboxyl group of complex 11.

The dialysis buffer includes urea because insulin has low solubility at neutral pH without urea, and urea allows a higher concentration of soluble insulin to be achieved. Likewise, the product is dialyzed against 2 mM NaOH because insulin has higher solubility in 2 mM NaOH than at neutral pH. If it is found that urea competes as a ligand to the Pt, it can be omitted from the dialysis buffer, but the volume of the reaction mixture should be increased to keep insulin soluble (proportionately lower concentrations of all components). Likewise, if 2 mM NaOH is found to have adverse effects for the platinum complex, then the product can be dialyzed against 20 mM sodium phosphate pH 7.4 at lower concentrations of the conjugate to keep it soluble.

### Example 2

In this Example, a dicarboxylate bidentate ligand is conjugated first to the protein, and then the modified protein is used to ligate a platinum complex to form the same insulin-platinum conjugate produced in Example 1.

CH(COO)₃Na₃ (30 μmoles) is dissolved with insulin (2 μmoles) in 3.4 ml of 20 mM sodium phosphate, pH 7.4, 6.5 M urea. The cross-linker 1-ethyl-3-[3-dimethylaminopropyl]carbodiimide hydrochloride (EDC) (300 μmoles) is freshly dissolved in 0.6 ml of the buffer, and added to the insulin solution. The solution is allowed to react for 2 hours at room temperature and then placed in a dialysis bag (3,500 m.w. cut-off). The solution is dialyzed three hours against 20 mM sodium phosphate, pH 7.4, 6.5 M urea, and then dialyzed overnight against 2 mM NaOH. The product is insulin with all amino groups modified to form —NHCOCH(COO)₂Na₂. This produces a bidentate dicarboxylate ligating group at the amino terminus and each lysine side chain of the protein.

Formation of insulin-platinum conjugate. The modified insulin is mixed with 4 mole equivalents of cis-[(NH₃)₂Pt(OH₂)₂](NO₃)₂ prepared as described in Example 1. The dicarboxylates substitute for water ligands to form conjugate 12

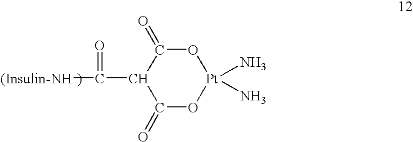

### Example 3

In this Example, complex 13 is prepared and conjugated to insulin.

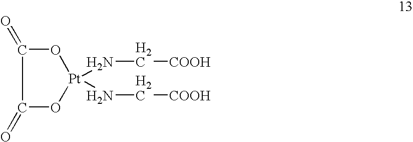

K₂PtI₄ (12 mmol) is formed as described in Example 1 and then mixed with 12-13 ml of 2.0 M glycine. After 30 minutes, the product cis(COOHCH₂NH₂)₂PtI₂ precipitate is filtered, washed with cold water, and dried.

Cis(COOHCH₂NH₂)₂PtI₂ is stirred with 2 mole equivalents of silver nitrate overnight. AgI, which precipitates, is filtered out. The filtrate contains cis-[(COOHCH₂NH₂)2Pt(OH₂)₂](NO₃)₂.

Cis-[(COOHCH₂NH₂)2Pt(OH₂)₂](NO₃)₂ is mixed with 1 mole equivalent of sodium oxalate. The solution is allowed to stand for 24 hours and then evaporated to dryness under vacuum. The product is complex 13. The product mixture also contains potassium nitrate.

Conjugation to insulin is done according to Example 1. The product is conjugate 14, with approximately three of complex 13 per mole of insulin.

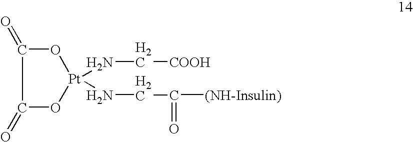

### Example 4

In this example, platinum is complexed to insulin by using the primary amino groups of lysine residues and the amino terminus as ligands to the platinum. K₂PtI₄ is incubated with insulin and ammonia at a mole ratio of 3 K₂PtI₄:1 insulin:3 NH₃. The mixture is stirred in aqueous solution at neutral pH overnight. Since there are three amino groups on insulin (one lysine, and two amino termini for the two polypeptides of mature insulin), this results in three complexed Pt per insulin with each Pt complexed on average by one amino group of insulin and one NH₃, along with two I.

The complex is then stirred with 2 mole equivalents of silver nitrate per mole of Pt overnight. AgI is filtered out. The filtrate is mixed with 1 mole equivalent of potassium oxalate and allowed to stand overnight. The product is conjugate 15.

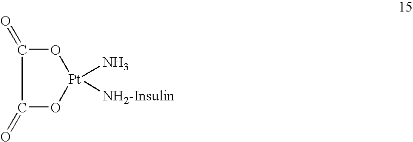

### Example 5

K₂PtI₄ prepared as in Example 1 is treated with 1 mole equivalent of ethylenediamine-N,N′-diacetic acid (EDDA). The platinum complex with EDDA is filtered, and washed with cold water. It is then stirred with 2 mole equivalents of silver nitrate in aqueous solution overnight. AgI is then filtered out. The filtrate contains EDDA-Pt(OH₂)₂(NO₃)₂. The EDDA-Pt(OH₂)₂(NO₃)₂ is mixed with one mole equivalent of potassium oxalate and allowed to stand for 24 hours and then evaporated to dryness under vacuum. The product is complex 16.

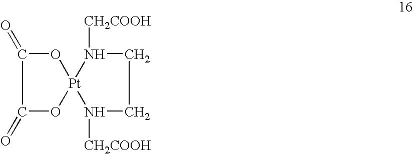

Complex 16 is conjugated to insulin via the free carboxyls of complex 16 forming amide bonds to amino groups of insulin as described in Example 1 to form conjugate 17.

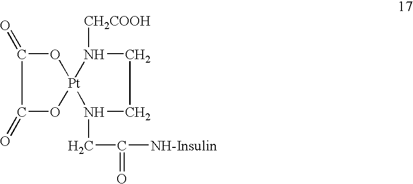

### Example 6

In this example, conjugate 17 is prepared by first conjugating EDDA to insulin by a procedure analogous to that described in Example 2 for conjugation of CH(COOH)₃ to insulin. This produces conjugate 18.

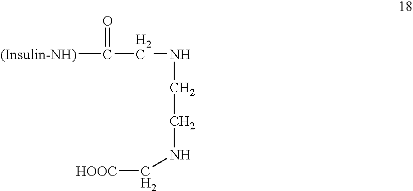

The conjugate is then reacted with K₂PtI₄ as in Example 5, and then with silver nitrate and potassium oxalate as described in Example 5 to form conjugate 17 with ligated Pt.

### REFERENCES

1. Momekov, G. et al., 2005, Novel approaches towards development of non-classical platinum-based antineoplastic agents: design of platinum complexes characterized by an alternative DNA-binding pattern and/or tumor-targeted cytotoxicity. Curr. Med. Chem. 12:2177-2191.
2. Cleare, M. J. et al. 1978. Anti-tumour platinum complexes: relationships between chemical properties and activity. Biochimie 60:835-850.
3. Harrison, R. C. et al. 1980. An efficient route for the preparation of highly soluble platinum(II) antitumour agents. Inorganica Chimica Acta 46:L15-L16.
4. Kidani, Y. et al. 1978. Antitumor activity of 1,2-diaminocyclohexane-platinum complexes against Sarcoma-180 ascites form. J. Med. Chem. 21:1315-1318.
5. Li, Jie Jack, et al. 2007. Modern Organic Synthesis in the Laboratory: a collection of standard experimental procedures. Oxford University Press, Oxford, UK.
6. Wyatt, Paul, and Stuart Warren. 2007. Organic Synthesis: strategy and control. John. Wiley, Hoboken, N.J., USA.
7. Tojo, Gabriel. 2006. Oxidation of primary alcohols to carboxylic acids: a guide to common practice. Springer Science and Business Media, Boston, Mass., USA.
8. Tojo, Gabriel. 2006. Oxidation of alcohols to aldehydes and ketones: a guide to common practice. Springer Science and Business Media, Boston, Mass., USA.
9. Taber, D. F. 2007. Organic synthesis: state of the art 2005-2007. John Wiley, Hoboken, N.J., USA.

All patent documents and other references cited are incorporated by reference.

What is claimed is:

1. A platinum complex of formula I or II 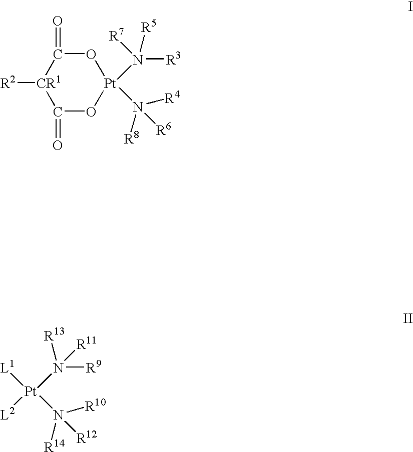
wherein in formula I
R¹ is H or (C₁-C₇)alkyl;
R² is COOH, NX₂, SH, HOOC—(C₁-C₁₀)alkyl, X₂N—(C₁-C₁₀)alkyl, HS—(C₁-C₁₀)alkyl, —CHO, OHC—(C₁-C₁₀)alkyl, or (C₁-C₆)alkyl-C(O)C(O)—(C₁-C₆)alkyl;
R³, R⁴, R⁵, R⁶, R⁷, and R⁸ are each independently H, (C₁-C₇)alkyl, or R³ and R⁴ together form (C₂-C₁₀)alkyl;
wherein in formula II
L¹ and L² are ligands selected from Cl⁻, formate, bicarbonate, NX₃, (C₁-C₁₀)alkyl-NX₂, and (C₁-C₁₀)alkyl-COO⁻, or L¹ and L² are together ⁻OOC—COO⁻, carboxy(C₁-C₁₀)alkyl-carboxy, X₂N—(C₁-C₁₀)alkyl-NX₂, or X₂N—(C₁-C₁₀)alkyl-carboxy;
R⁹, R¹⁰, R¹¹, R¹², R¹³, and R¹⁴ are each independently H, X₂N—(C₁-C₁₀)alkyl, HOOC—(C₁-C₁₀)alkyl, HS—(C₁-C₁₀)alkyl, —CHO, OHC—(C₁-C₁₀)alkyl, or (C₁-C₆)alkyl-C(O)C(O)—(C₁-C₆)alkyl; or R⁹ and R¹⁰ together are (C₂-C₁₀)alkyl, HOOC—(C₂-C₁₀)alkyl, X₂N—(C₂-C₁₀)alkyl, HS—(C₂-C₁₀)alkyl, OHC—(C₂-C₁₀)alkyl, or (C₁-C₆)alkyl-C(O)C(O)—(C₁-C₆)alkyl;
wherein at least one of R⁷, R⁸, R⁹, and R¹⁰ is HOOC—(C₁-C₁₀)alkyl, X₂N—(C₁-C₁₀)alkyl, HS—(C₁-C₁₀)alkyl, —CHO, OHC—(C₁-C₁₀)alkyl, or (C₁-C₆)alkyl-C(O)C(O)—(C₁-C₆)alkyl;
wherein L¹ is optionally R¹³ and L² is optionally R¹⁴;
wherein each X is independently H or (C₁-C₁₀)alkyl;
wherein each alkyl is optionally saturated or unsaturated, and straight chain, branched, or cyclic, optionally interrupted with —NH—, —O—, —S—, or ═N—, and optionally substituted with OH, halo, or oxo.

2. The platinum complex of claim 1 wherein the complex is of formula II, R¹¹ and R¹² are each H, and R⁹ and R¹⁰ together form —(C₂-C₃)alkyl- optionally substituted with carboxy, amino, mercapto, carboxy(C₁-C₄)alkyl, amino(C₁-C₄)alkyl, or mercapto(C₁-C₄)alkyl; and R₁₃ and R₁₄ are independently H, carboxy(C₁-C₄)alkyl, amino(C₁-C₄)alkyl, or mercapto(C₁-C₄)alkyl.

3. The platinum complex of claim 1 wherein the complex is of formula II and R⁹ and R¹⁰ together form —(C₂-C₃)alkyl- optionally substituted with carboxy or carboxy(C₁-C₄)alkyl; and R¹¹-R¹⁴ are each independently H, (C₁-C₄)alkyl, or carboxy(C₁-C₄)alkyl; wherein at least one of R⁹-R¹⁴ is carboxy(C₁-C₆)alkyl.

4. The platinum complex of claim 1 wherein the complex is of formula I and R³ and R⁴ together form (C₂-C₃)alkyl.

5. A polypeptide-platinum conjugate of formula III or IV 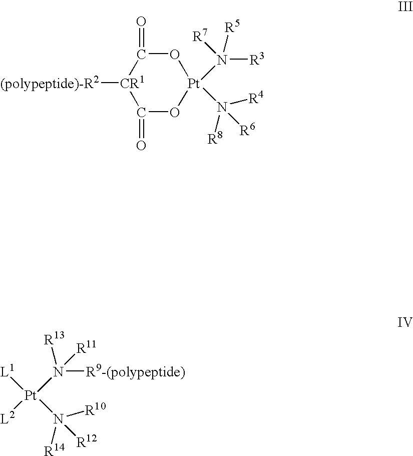
wherein in formula III
R¹ is H or (C₁-C₇)alkyl;
R² is a linker moiety of 1-100 atoms;
R³, R⁴, R⁵, R⁶, R⁷, and R⁸ are each independently H, (C₁-C₇)alkyl, or R³ and R⁴ together form (C₂-C₁₀)alkyl;
wherein in formula IV
L¹ and L² are ligands selected from Cl⁻, formate, bicarbonate, NX₃, (C₁-C₁₀)alkyl-NX₂, and (C₁-C₁₀)alkyl-COO⁻, or L¹ and L² are together ⁻COO—COO⁻, carboxy-(C₁-C₁₀)alkyl-carboxy, X₂N—(C₁-C₁₀)alkyl-NX₂, or X₂N—(C₁-C₁₀)alkyl-carboxy;
R⁹ is a linker moiety of 1-100 atoms;
R¹⁰, R¹¹, R¹², R¹³, and R¹⁴ are each independently H, (C₁-C₁₀)alkyl, X₂N—(C₁-C₁₀)alkyl, HOOC—(C₁-C₁₀)alkyl, or HS—(C₁-C₁₀)alkyl; or R¹¹ and R¹⁰ together are (C₂-C₁₀)alkyl, HOOC—(C₂-C₁₀)alkyl, X₂N—(C₂-C₁₀)alkyl, or HS—(C₂-C₁₀)alkyl; or R⁹ and R¹⁰ together are a linker moiety of 1-100 atoms;
wherein L¹ is optionally R¹³ and L² is optionally R¹⁴;
wherein each X is independently H or (C₁-C₁₀)alkyl;
wherein each alkyl is optionally saturated or unsaturated, and straight chain, branched, or cyclic, optionally interrupted with —NH—, —O—, —O—, or ═N—, and optionally substituted with OH, halo, or oxo.

6. The conjugate of claim 5 wherein the polypeptide is a ligand to the insulin, IGF-1, or EGF receptors.

7. The conjugate of claim 5 wherein the linker moiety of R² or R⁹ comprises a —C(═O)— or —NH— portion of an amide bond linking to the residue of an amine or carboxy group of the protein or peptide.

8. The conjugate of claim 6 wherein the polypeptide is a ligand to the EGF receptor, the ligand comprising a polypeptide sequence selected from the group consisting of residues 2-54 of SEQ ID NO:1, residues 40-89 of SEQ ID NO:2, residues 101-184 of SEQ ID NO:3, residues 63-148 of SEQ ID NO:4, residues 32-111 of SEQ ID NO:5, and E4T.

9. The conjugate of claim 6 wherein the polypeptide is a ligand to the IGF-1 receptor, the ligand comprising a polypeptide sequence selected from the group consisting of SEQ ID NOS:8-14.

10. A method of making a polypeptide-platinum conjugate comprising:
forming a platinum complex of claim 1; and
reacting the platinum complex of claim 1 with a linker reactant and a polypeptide to form a polypeptide-platinum conjugate of claim 5.

11. A method of making a polypeptide-platinum conjugate comprising:
reacting a platinum complex with a polypeptide-bidentate ligand conjugate of formula VI 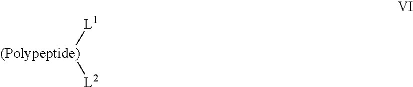
to form a polypeptide-platinum conjugate of formula VII 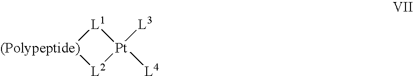
wherein L¹-L⁴ are each ligands; wherein the polypeptide-platinum conjugate of formula VII is a polypeptide-platinum conjugate of claim 5.

12. The method of claim 11 wherein the method comprises:
reacting a platinum complex of formula V with a polypeptide-bidentate ligand conjugate of formula VI 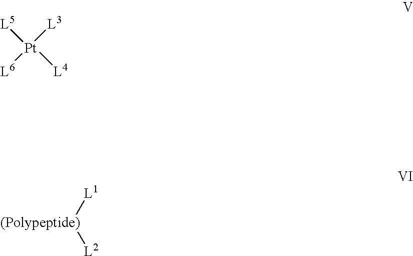
to form a polypeptide-platinum conjugate of formula VII 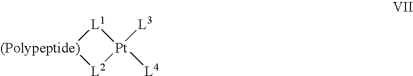
wherein L¹-L⁶ are each ligands; wherein the polypeptide platinum conjugate of formula VII is a polypeptide-platinum conjugate of claim 21.

13. The method of claim 11 wherein the polypeptide-bidentate ligand conjugate of formula VI is a conjugate of formula VIII 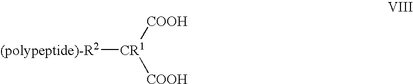
wherein R¹ is H or (C₁-C₇)alkyl and R² is a linker moiety of 1-100 atoms; wherein each alkyl is optionally saturated or unsaturated, and straight chain, branched, or cyclic, optionally interrupted with —NH—, —O—, —S—, or ═N—, and optionally substituted with OH, halo, or oxo.

14. The method of claim 11 wherein the polypeptide-bidentate ligand conjugate of formula VI is a conjugate of formula IX 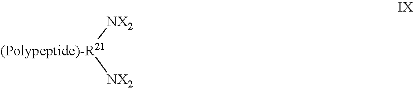
wherein R²¹ is (C₁-C₂)alkyl, optionally substituted with (C₁-C₇)alkyl, and each X is independently H or (C₁-C₁₀)alkyl;
wherein each alkyl is optionally saturated or unsaturated, and straight chain, branched, or cyclic, optionally interrupted with —NH—, —O—, —S—, or ═N—, and optionally substituted with OH, halo, or oxo.

15. A polypeptide-platinum complex of formula X 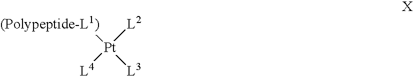
wherein L¹ is an amino, caboxy, or sulfhydryl group of the polypeptide that is a ligand to the Pt, and L²-L⁴ are ligands.
